(19) United States
(12) Patent Application Publication
(10) Pub. No.: US 2023/0067805 A1
(43) Pub. Date: Mar. 2, 2023

(54) PEG-MODIFIED POLYPEPTIDE CAPABLE OF INHIBITING GP96, PREPARATION METHOD THEREFOR AND USE THEREOF

(71) Applicant: BEIJING COMINGHEALTH BIO TEC CO., LTD., Beijing (CN)

(72) Inventors: Jun HU, Beijing (CN); Songdong MENG, Beijing (CN); Zhe YANG, Beijing (CN); Biao WU, Beijing (CN); Bo YANG, Beijing (CN)

(21) Appl. No.: 17/758,535

(22) PCT Filed: Oct. 22, 2020
(86) PCT No.: PCT/CN2020/122712

§ 371 Date: Jul. 8, 2022

(30) Foreign Application Priority Data
Jan. 10, 2020 (CN) 202010024127.2

(51) Int. Cl. A61K 38/17; A61K 47/60; A61P 35/00; C07K 1/113

(52) U.S. Cl. CPC A61K 38/17; A61K 47/60; A61P 35/00; C07K 1/113

(57) ABSTRACT

The present invention belongs to the field of biomedicine, and provided is a PEG-modified polypeptide capable of inhibiting gp96, which comprises PIBC linked by a covalent bond and PEG having an average molecular weight of 20,000-40,000. Further provided is a method for preparing the PEGylated polypeptide, a drug or preparation comprising the PEGylated polypeptide, and a use of the PEGylated polypeptide.

### TECHNICAL FIELD

The invention relates to the field of biomedicines, in particular to a PEG-modified polypeptide capable of inhibiting gp96, a preparation method and use thereof.

### BACKGROUND ART

Breast cancer is a cancer type having a highest incidence rate among women in China, and the incidence rate is progressively increasing at a rate of 3% per year. Thus, the cancer becomes a cancer with a mortality rate that is most quickly increasing in cities. Although there are a great variety of medicines for the breast cancer, they also have some limitations, wherein the most remarkable limitation is the lack of medicines for treating triple negative breast cancer. The triple negative breast cancer (TNBC) refers to a breast cancer in which Estrogen Receptors (ER), Progestogen Receptors (PR) and Human Epidermal Growth Factor Receptors 2 (Her-2) are negative, and it accounts for about 15%-20% of the pathological types of the breast cancer, being a highly malign tumor. At present, for TNBC, the treating means and medicines are limited, and treatment targets and target medicines are lack. Thus, it is in the urgent need to develop novel potential targets.

With immunohistochemistry test to tumor tissues in 80 patients suffering from breast cancers, it was found that about 70% of the patents showed highly expressed heat shock protein gp86 on the cytomembrane of the breast cancer, including most of the patients suffering from the triplet negative breast cancer, whilst the surface of normal cytomembrane would not express the gp96. Hence, the cytomembrane gp96 can be used as a molecular marker of the triplet negative breast cancer and as a latent treatment target. On this basis, a polypeptide containing an α-helical sequence, with a code PIBC, is designed based on the amino acid sequence and spatial conformation of the gp96. The polypeptide can specifically bind to the gp96, to block intermolecular motif rearrangements and conformation changes of the gp96, and further interfere interactions of the cytomembrane gp96 with HER-2, uPAR and ER-a36 and cause endocytoses and degradations of these tumor proteins (Chinese patent ZL 201110159487.4). By both cell experiments in vitro and tumor-bearing mouse experiments in vivo, it is found that the polypeptide medicine can effectively inhibit the growth of the triple negative breast cancer, promote apoptosis whilst inhibiting tumor invasions and metastasis, and it is indicated the polypeptide can be used as a candidate medicine for targeting the triple negative breast cancer.

While the polypeptide has a specific action site, it has drawbacks of strong irritation, low solubility and high immunogenicity, and it is prone to be degraded by proteases and cleaned up by kidney, and these drawbacks will seriously restrict the clinical application of the polypeptide.

### SUMMARY OF THE INVENTION

One of the technical problems to be solved by the invention is how to reduce the toxicity and irritation of the PIBC, improve the hemolytic property of the PIBC and prolong the half-life period of the PIBC so as to ensure the PIBC to be safer and more effective. The inventors find that the above-described effects can be achieved by modifying the PIBC with polyethylene glycol (PEG), and thereby the following invention is provided:

### PEGylated Polypeptide

In one aspect, the present application provides a PEGylated polypeptide comprising a PIBC and a PEG having an average molecular weight (a number average molecular weight (Mn)) of about 20000-40000, the PIBC being covalently linked to the PEG, wherein the PIBC is selected from:

A1) a polypeptide having an amino acid sequence shown by SEQ ID NO. 1; and
A2) a polypeptide derived from A1) and having the same functions as A1), with an amino acid sequence, as compared with the amino acid sequences shown by SEQ ID NO. 1, with replacements (e.g., conservative replacements) and/or deletions and/or additions of one or more (e.g., 1-10 or 1-5 or 1-3) amino acid residues or with at least 60%, 80%, 85%, 90%, 95%, 96%, 97%, 98% or 99% identity.

In certain embodiments, the PEG is linked to the N-end or C-end of the PIBC.

In certain embodiments, the PEG is a linear PEG or a branched PEG.

In certain embodiments, the PEG has an average molecular weight of about 20000-25000, 25000-30000, 30000-35000, or 35000-40000, e.g., about 20000, 21000, 22000, 23000, 24000, 25000, 26000, 27000, 28000, 29000, 30000, 31000, 32000, 33000, 34000, 35000, 36000, 37000, 38000, 39000, or 40000. In the PEGylated polypeptide of the invention, the PIBC and the PEG can be either covalently linked via a linking group, or directly linked via a covalent bond.

In certain embodiments, the PEGylated polypeptide has the following structure:

R—(CH₂CH₂O)ₙ-linker-PIBC;

wherein n is the polymerization degree of the PEG, and the n meets the condition that the PEG has the molecular weight of about 20000-40000; R is the end group of the PEG, such as methoxyl group; “linker” is a linking group, e.g.,

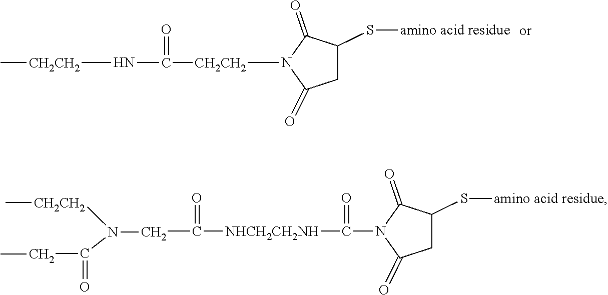

the amino acid residue being an amino acid (e.g., cysteine (Cys)) residue with a thiol group.

In certain embodiments, the PEGylated polypeptide has a structure represented by formula I:

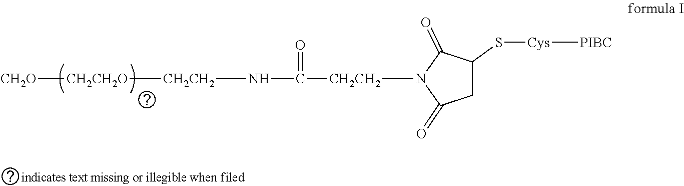

In the formula I, n is the polymerization degree of the PEG, and the n meets the condition that the PEG has a molecular weight of about 20000-40000.

In certain embodiments, in the formula I, Cys is linked to a PIBC via a peptide bond formed with the carboxyl group of the Cys and the amino group at the N end of the PIBC, or via a peptide bond formed with the amino group of the Cys and the carboxyl group at the C end of the PIBC.

In certain embodiments, the covalent linking is accomplished by performing a Michael addition reaction with Reactant 1 and Reactant 2, wherein the Reactant 1 is a PEG linked to maleimide at one end, and the Reactant 2 is a PIBC with a thiol-containing amino acid residue at the N- or C-end. The Michael addition reaction occurs between the maleimide and the thiol group.

In certain embodiments, the Reactant 1 has a structure represented by formula II:

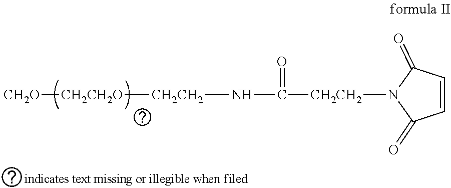

In the formula II, n is the polymerization degree of the PEG, and the n meets the condition that the PEG has the molecular weight of 20000-40000. The Reactant 1 may be referred to as methoxy polyethylene glycol maleimide (mPEGxMaI, where x denotes the average molecular weight of the PEG; MaI denotes maleimide, the maleimide modification being at one end of the PEG; m denotes a methoxy group).

In certain embodiments, in the Reactant 2, the thiol-containing amino acid residue is a cysteine residue. In certain embodiments, the Reactant 2 is a polypeptide with an amino acid sequence shown by SEQ ID NO. 2 or SEQ ID NO. 3.

### Method for Preparing PEGylated Polypeptide

In one aspect, the present application provides a method of preparing a PEGylated polypeptide of the invention, comprising the step of covalently linking a PIBC and a PEG having an average molecular weight of about 20000-40000.

In certain embodiments, the method comprises a step of performing a Michael addition reaction with Reactant 1 and Reactant 2; Reactant 1 is a PEG linked to maleimide at one end, and Reactant 2 is a PIBC with a thiol-containing amino acid residue at the N- or C-end. The Michael addition reaction occurs between the maleimide and the thiol group.

In certain embodiments, the Reactant 1 or the Reactant 2 is defined as above.

In certain embodiments, the Michael addition reaction is performed at the condition with a pH of about 7.2-7.6. In certain embodiments, the Michael addition reaction is performed in a NaH₂PO₄ buffer solution.

In certain embodiments, the Michael addition reaction is performed at room temperature (e.g., 20-30° C.).

In certain embodiments, the Reactant 1 is excessive relative to the Reactant 2. In certain embodiments, the initial molar ratio of Reactant 1 to Reactant 2 is about 2-10: 1.

In certain embodiments, the method further comprises a step of subjecting the reaction product to purification. In certain embodiments, the purification is accomplished by chromatography (e.g., ion exchange chromatography or high performance liquid chromatography).

### Pharmaceutical Compositions, Formulations and Uses

In one aspect, the present application provides pharmaceutical compositions comprising the PEGylated polypeptide of the invention. In certain embodiments, the pharmaceutical compositions are used to treat and/or prevent diseases associated with gp96 protein overexpression in a subject.

The pharmaceutical compositions of the invention may further comprise one or more pharmaceutical carriers. Useful pharmaceutical carriers in the invention include, but are not limited to, fillers, diluents, binders, wetting agents, disintegrants, lubricants, surfactants, preservatives, colorants, flavors, fragrances, effervescent agents, emulsifiers, flocculants, deflocculants, bacteriostats, solubilizers. In certain embodiments, the pharmaceutical carriers are selected from the group consisting of ion exchangers, alumina, aluminum stearate, lecithin, serum proteins (e.g., human serum protein), glycerol, sorbic acid, potassium sorbate, water, protamine sulfate, disodium hydrogen phosphate, potassium hydrogen phosphate, sodium chloride, zinc salts, colloidal silica, magnesium trisilicate, polyvinylpyrrolidone, cellulosic materials, polyethylene glycol, sodium carboxymethylcellulose, polyacrylates, beeswax, polyethylene-polyoxypropylene block polymers, lanolin, and any combination thereof.

The pharmaceutical compositions of the present invention may be formulated into a variety of suitable dosage forms, including, but not being limited to, oral dosage forms, injectable dosage forms (e.g., suitable for subcutaneous injection, intramuscular injection, or intravenous injection), inhalant dosage forms, mucosal administration dosage forms, or topical administration dosage forms. In certain embodiments, the pharmaceutical compositions are formulated into oral dosage forms, for example, such as tablets, capsules, granules, oral solutions, oral suspensions, pellets, or mini-tablets.

In one aspect, the present application provides use of the PEGylated polypeptide of the invention in the manufacture of a medicine for the treatment and/or prevention of a disease associated with gp96 protein overexpression (e.g., a tumor) in a subject.

In one aspect, the present application provides formulations containing the PEGylated polypeptide of the invention. In some embodiments, the formulation is used, to bind to a gp96 protein, to inhibit proliferation and/or growth and/or invasion of a tumor cell, to promote apoptosis of a tumor cell, and/or to inhibit tumor growth.

In one aspect, the present application provides use of the PEGylated polypeptide of the invention in the manufacture of a formulation, wherein the formulation is used to bind to a gp96 protein, to inhibit proliferation and/or growth and/or invasion of a tumor cell, to promote apoptosis of a tumor cell, and/or to inhibit tumor growth.

The formulation of the invention may be administered in vivo or in vitro; for example, the formulation is administered into the body of a subject to bind gp96 protein in the body of the subject, thereby to inhibit proliferation and/or growth and/or invasion of a tumor cell in the body of the subject, promote apoptosis of a tumor cell in the body of the subject, and/or inhibit tumor growth in the body of the subject; alternatively, the formulation is administered to gp96 protein in vitro to bind the gp96 protein in vitro; alternatively, the formulation is administered to a cell in vitro (e.g., a cell line or a cell from a subject, e.g., a tumor cell) to inhibit proliferation and/or growth and/or invasion of a tumor cell in vitro, and/or to promote apoptosis of a tumor cell in vitro.

In one aspect, the present application provides a method of treating and/or preventing a disease associated with gp96 protein overexpression (e.g., a tumor) in a subject, comprising administering a therapeutically and/or prophylactically effective amount of the PEGylated polypeptide or the pharmaceutical composition of the invention to the subject in need.

In one aspect, the present application provides a method of inhibiting proliferation and/or growth and/or invasion of a tumor cell, promoting apoptosis of a tumor cell, and/or, inhibiting tumor growth, comprising administering the PEGylated polypeptide or the formulation of the invention to the tumor cell or tumor. In certain embodiments, the tumor cell is present in the body of a subject, and the method is performed in the body of the subject. In certain embodiments, the tumor cell is present in vitro, and the method is performed in vitro. The methods may be used either for prophylactic or therapeutic purpose, or for non-prophylactic or therapeutic purpose (e.g., scientific research).

In the above embodiments of the invention, the gp96 protein may be in vitro or in the body of a subject.

In the above embodiments of the invention, the tumors include, but are not limited to, brain tumor, lung cancer, squamous cell carcinoma, bladder cancer, stomach cancer, ovarian cancer, peritoneal cancer, pancreatic cancer, breast cancer, head and neck cancer, cervical cancer, endometrial cancer, rectal cancer, liver cancer, kidney cancer, esophageal adenocarcinoma, esophageal squamous cell carcinoma, prostate cancer, cancer of the female reproductive tract, carcinoma in situ, lymphoma, neurofibroma, thyroid cancer, bone cancer, skin cancer, brain cancer, colon cancer, testicular cancer, gastrointestinal stromal tumor, prostate tumor, mast cell tumor, multiple myeloma, melanoma, glioma, or sarcoma. In certain embodiments, the tumor is breast cancer, e.g., triple negative breast cancer.

In the above embodiments of the invention, the tumor cells include, but are not limited to: brain tumor cells, lung cancer cells, squamous cell carcinoma cells, bladder cancer cells, stomach cancer cells, ovarian cancer cells, peritoneal cancer cells, pancreatic cancer cells, breast cancer cells, head and neck cancer cells, cervical cancer cells, endometrial cancer cells, rectal cancer cells, liver cancer cells, kidney cancer cells, esophageal adenocarcinoma cells, esophageal squamous cell carcinoma cells, prostate cancer cells, cancer cells of the female genital tract, cancer cells in situ, lymphoma cells, neurofibroma cells, thyroid cancer cells, bone cancer cells, skin cancer cells, brain cancer cells, colon cancer cells, testicular cancer cells, gastrointestinal stromal tumor cells, prostate tumor cells, mast cell tumor cells, multiple myeloma cells, melanoma cells, glioma cells, or sarcoma cells.

In certain embodiments, the breast cancer cell is SKBr3 or MDA-MB-231.

In the above embodiments of the invention, the subject may be a mammal, such as bovine, equine, porcine, canine, feline, rodent, primate; among these, the particularly preferred subject is human.

### Term Definitions

In the invention, unless otherwise specified, scientific and technical terms used here have the meanings that are commonly understood by those skilled in the art. Moreover, all the laboratory operation steps referred to here are conventional steps that are widely used in corresponding fields. Meanwhile, in order to better understand the invention, the definitions and explanations for related terms are provided below.

In the invention, the gp96 protein refers to a heat shock protein (also called GRP94) with a molecular weight of about 96KD, which exists in endoplasmic reticulum of eukaryotic cells. The amino acid sequence of the gp96 protein is known to those skilled in the art and it can be found in various public databases (e.g., GenBank database, Genbank Accession number AY 040226). An exemplary amino acid sequence of the wild-type gp96 protein is shown by SEQ ID NO. 4. Thus, in the invention, when referring to the sequence of the gp96 protein, it is described by using the sequence shown by SEQ ID NO. 4. However, it is understood by those skilled in the art that mutations or variations (including, but being not limited to, replacements, deletions and/or additions) can be naturally generated or artificially introduced in SEQ ID NO. 4 without affecting the biological characteristics of the gp96 protein. Thus, in the invention, the term “gp96 protein” is intended to encompass all polypeptides and variants in this kind, including the polypeptide shown by SEQ ID NO. 4 and natural or artificial variants thereof, which retain the biological characteristics of the gp96 protein.

In the invention, the PIBC (Peptide Inhibitor for Breast Cancer) is a polypeptide capable of binding to gp96 protein. The polypeptide contains an α-helical sequence, and it can specifically bind to the gp96, to block intramolecular gene sequence rearrangements and conformation changes of the gp96, and further to interfere the interactions of cytomembrane gp96 with HER-2, uPAR or ER-a36. An exemplary amino acid sequence of the PIBC is as shown by SEQ ID NO. 1. In the invention, the term “PIBC” is intended to encompass variants of the PIBC. The term “variants” refers to polypeptides with an amino acid sequence, as compared with the amino acid sequence of the PIBC, differing in by one or more (e.g., 1-10 or 1-5 or 1-3) amino acids (e.g. conservative replacements of amino acids), or having at least 60%, 80%, 85%, 90%, 95%, 96%, 97%, 98% or 99% identity, and having the same functions as the PIBC. The term “functions” may be one or more of the following functions: (i) being capable of specifically binding to gp96, (ii) being capable of blocking the intramolecular gene sequence rearrangement and conformational changes of gp96, and (iii) being capable of interfering interactions of cytomembrane gp96 with HER-2, uPAR or ER-a36.

As used here, the term “identity” refers to the match in sequences between two polypeptides or between two nucleic acids. When some position in two sequences that are compared is occupied by the same base or amino acid monomer subunit (e.g., some position in each of two DNA molecules is occupied by adenine, or some position in each of two polypeptides is occupied by lysine), the respective molecules at the position are identical. The “percent identity” between two sequences is a function that the number of the matching positions shared by the two sequences is divided by the number of the positions that are compared, and the result multiplies with 100. For example, if there are six matching positions in 10 positions of two sequences, the two sequences have a 60% identity. For example, the DNA sequences CTGACT and CAGGTT share a 50% identity (in total six positions, 3 positions are matching positions). Typically, two sequences are compared in a manner to produce a maximum identity. This comparison may be performed, for example, by a method according to Needleman et al. (1970) J. Mol. Biol. 48: 443-453 that can be conveniently accomplished by a computer program, such as Align progress (DNAstar, Inc.). Also, an algorithm, E. Meyers and W. Miller (Compout. Appl biosci., 4:11-17(1988)), that has been integrated into the ALIGN program (version 2.0) may be used to determine the percent identity in the sequences between two amino acids with a PAM120 weight residue table, a gap length penalty score of 12 and a gap length penalty score of 4. Furthermore, an algorithm, Needleman and Wunsch (J Mol biol. 48:444-453 (1970)), that is integrated into GAP program of GCG software package (available at www.gcg.com) may be used to determine percent identity in the sequences between two amino acids with a Blossum 62 matrix or a PAM250 matrix, a gap weight of 16, 14, 12, 10, 8, 6, or 4 and a length weight of 1, 2, 3, 4, 5, or 6.

As used here, the term “conservative replacement” means an amino acid replacement that does not adversely affect or alter biological activities of protein/polypeptide comprising amino acid sequences. For example, a conservative replacement may be introduced by standard techniques known in the art, e.g., site-directed mutagenesis and PCR-mediated mutagenesis. The conservative amino acid replacements include those replacements in which an amino acid residue is replaced with an amino acid residue having a similar side chain, e.g., a replacement that is performed by replacing a corresponding amino acid residue with a residue physically or functionally similar thereto (e.g., to have similar sizes, shapes, charges, chemical properties, including abilities to form covalent or hydrogen bonds, and the like). Families of amino acid residues having similar side chains have been defined in the art. These families include amino acids with basic side chains (e.g., lysine, arginine, and histidine), acidic side chains (e.g., aspartic acid, and glutamic acid), uncharged polar side chains (e.g., glycine, asparagine, glutamine, serine, threonine, tyrosine, cysteine, and tryptophan), nonpolar side chains (e.g., alanine, valine, leucine, isoleucine, proline, phenylalanine, and methionine), β-branched side chains (e.g., threonine, valine, and isoleucine), and aromatic side chains (e.g., tyrosine, phenylalanine tryptophan, and histidine). Thus, it is preferred to replace a corresponding amino acid residue with the other amino acid residue from the same side chain family. Methods for identifying the amino acid conservative replacement are well known in the art (see, e.g., Brummell et al, biochem. 32: 1180-1187 (1993); Kobayashi et al Protein Eng. 12 (10): 879-884 (1999); and Burks et al Proc. Natl Acad. Set USA 94:412-417 (1997), which are incorporated herein by reference).

As used here, the term “about” should be understood by one of skill in the art and varies to some extent depending on the contexts in which it is used. If the meanings are not clear for one of skill in the art according to the contexts in which the term is applied, the meaning of the term “about” refers to a deviation of no more than ±10% of specific values or ranges.

As used here, the term “effective amount” refers to an amount sufficient to achieve or at least partially achieve desired effects. For example, a therapeutically effective amount refers to an amount sufficient to cure or at least partially prevent a disease and complications thereof in a patient suffering from the disease. It is well within the ability of one of skill in the art to determine such effective amounts. For example, an effective amount for therapeutic use will depend on severities of the disease to be treated, general states of patient's own immune system, general conditions of patients, e.g., age, weight and sex, administration modes of medicines, concurrently administered other treatments, and the like.

An amount of a medicine administered to a subject depends on types and serveries of the disease or conditions and characteristics of the subject, such as general health, age, sex, body weight and tolerance to the drug, and further on types of formulations and administration modes of medicines, and administration periods or intervals. One of skill in the art can determine an appropriate dose based on these and other factors.

### Advantageous Effects

It is proved with experiments that the PEG-modified PIBC has an affinity to gp96 protein, and it not only can remarkably inhibit proliferation (growth) and invasion of tumor cells, remarkably promote apoptosis of tumor cells, and effectively inhibit tumor growth caused by tumor cells, more importantly, it can remarkably reduce irritation of the PIBC, prolong half-life in vivo of the polypeptide, and finally remarkably improve the druggability of the PIBC. The above results show that the PEG-modified PIBC can be used as a medicine for treating tumors.

### ILLUSTRATIONS TO THE DRAWINGS

FIG. 1 shows the mass spectrometry identification results of the PEGylated polypeptide mPEG₂₀₀₀₀CY in Example 1.

FIG. 2 shows the mass spectrometry identification results of the PEGylated polypeptide mPEG₄₀₀₀₀CY in Example 2.

FIG. 3 shows the mass spectrum identification result of the PEGylated polypeptide mPEG₂₀₀₀₀LC in Example 3.

FIG. 4 shows the mass spectrum identification result of the PEGylated polypeptide mPEG₄₀₀₀₀LC in Example 4.

FIG. 5 shows the identification result of secretory human heat shock protein gp96 expressed by insect cell expression system in Example 5; 1: molecular weight standard; 2: purified gp96 protein; 3: western blot results of gp96 protein.

FIG. 6 shows the tumor inhibition rates of the PIBC and mPEG₂₀₀₀₀CY treated groups in Example 9.

### DETAILED DESCRIPTIONS TO THE INVENTION

The invention is further described in detail below with reference to specific embodiments, and the examples are given only for illustrating the invention but not for limiting the scope of the invention. The test methods in the following examples, unless otherwise specified, each are all conventional ones. Materials, reagents, instruments and the like used in the following examples, unless otherwise specified, each are commercially available. For the quantitative tests in the following examples, each is provided with three repeated tests and the result takes the average value thereof.

Preparation of PBS buffer solution: 8 g of NaCl, 0.2 g of KCl, 3.625 g of Na₂HPO₄.12H₂O, 0.24 g of KH₂PO₄ were added, water was added to obtain 1 L of solution, and the pH of the solution was adjusted to pH to 7.3.

Preparation of 5 mM of Na₂HPO₄ solution: deionized water was added to 1.7907 g of Na₂HPO₄.12H₂O to obtain 1 L of solution.

Preparation of 5 mM of NaH₂PO₄ solution: deionized water was added to 0.78 g of NaH₂PO₄.2H₂O to obtain 1 L of solution.

Mass spectra were analyzed on a VG PLATFORM mass spectrometer by injecting the samples according to MALDI-TOF technique.

In the following examples, unless otherwise specified, the ratio of liquid to liquid is a ratio of volume to volume; the ratio of solid to liquid is a ratio of amount of material in mmol to volume in mL; the ratio of solid to solid is a ratio of mass to mass.

In the following examples, unless otherwise specified, the room temperature is specifically controlled at temperatures in a range of 20-30° C., including 20° C. and 30° C.

### Example 1: Preparation of PEGylated Polypeptide mPEG₂₀₀₀₀CY

### (I) Obtaining of a Polypeptide

A polypeptide obtained by adding cysteine to the N-end of the PIBC shown by SEQ ID NO. 1 in the sequence table was named as “CY”; the amino acid sequence of the peptide CY was shown by SEQ ID NO. 2 in the sequence table; the polypeptide CY shown by SEQ ID NO. 2 was synthesized by GL Biochem (Shanghai) Ltd.

### (II) PEGylation Modifications of the Polypeptide

Raw materials: mPEGxMaI (methoxy polyethylene glycol maleimide, wherein x is the average molecular weight of the PEG; MaI denotes maleimide, the maleimide modification being at one end of the PEG; and m denotes methoxy), having a chemical structure represented by formula II:

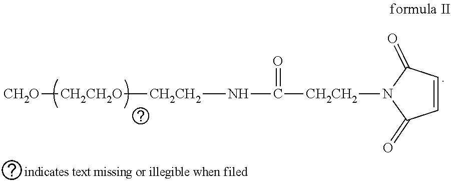

The mPEGxMaI (x=20000, i.e., the PEG has an average molecular weight of 20000, and mPEG₂₀₀₀₀MaI is a product of Beijing JenKem Technology Co., Ltd.), via a Michael addition reaction, was reacted with the thiol group in the N-end amino acid cysteine of the polypeptide CY, to produce the mPEG₂₀₀₀₀CY.

1. Synthesis of mPEG₂₀₀₀₀CY

A mixture of 80 mg (0.004 mmol) of the mPEG₂₀₀₀₀MaI and 10 mg (0.002 mmol) of the polypeptide CY was dissolved in 10 mL of a 5 mM NaH₂PO₄ buffer solution, the pH of the reaction solution was adjusted to 7.2 with the 5 mM Na₂HPO₄ solution. The reaction was monitored by HPLC until the polypeptide was completely reacted.

2. Purification and Characterization of PEGylated Polypeptide mPEG₂₀₀₀₀CY

HiTrap SP FF (1 mL) was used to purify the mPEG₂₀₀₀₀CY in a large quantity. The eluent included a mobile phase solution A1 and a mobile phase solution B1, in which the solution A1 consisted of solutes and a solvent, the solvent being 20 mM Tris-HCl (pH 7.4), and the solutes and the concentrations thereof being 1 mM EDTA.2Na and 0.01% (mass percent) NaN₃ respectively; the solution B1 consisted of solutes and a solvent, the solvent being 20 mM Tris-HCl (pH7.4), and the solutes and the concentrations thereof being 1000 mM NaCl, 1 mM EDTA.2Na and 0.01% (mass percentage) NaN₃ respectively. The elution conditions were described below: a mixed solution of the solution A1 and the solution B1, with the volume percentages of 80% and 20% respectively, was used to make the baseline stable, and then a mixed solution of the solution A1 and the solution B1, with the volume percentages of 70% and 30% respectively, was used to elute and collect the samples. The collected samples were centrifugation concentrated to a volume of 500 μl by a Millipore ultrafiltration centrifuge tube (10 KD) at 4° C., with a speed of 3500 r/min. The concentrated samples were desalted by HiTrap Desalting (5 mL). The PEGylated purified product of polypeptide mPEG₂₀₀₀₀CY in a fluffy state was obtained after removing the solvent by freeze-drying.

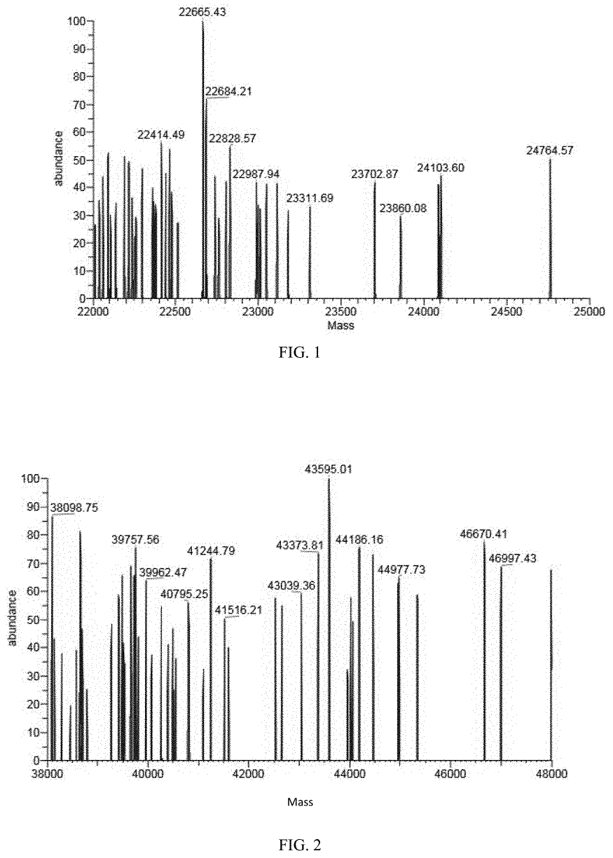

The chemical structure of the mPEG₂₀₀₀₀CY was characterized by MALDI-TOF mass spectrum, and result of the mass spectrum characterization of the mPEG₂₀₀₀₀CY was shown in FIG. 1. The structural formula of mPEG₂₀₀₀₀CY is shown by formula I, wherein the Cys and the PIBC were linked by a peptide bond formed by the carboxyl group of the Cys and the amino group of the N-end amino acid residue of the PIBC.

The purity of the mPEG₂₀₀₀₀CY was given by an analytical high performance liquid chromatograph (flow rate: 1 mL/min). The model of the analytical high performance liquid chromatograph was Angilent 1200, and the model of chromatographic column was Angilent Eclipse XDB-C18 Analytical, 5 μm, 4.6×150006 Dm. The operation of chromatograph was described below: a linear gradient elution, wherein the eluent consisted of a mobile phase solution A2 and a mobile phase solution B2, the mobile phase solution A2 being a trifluoroacetic acid aqueous solution with trifluoroacetic acid in a volume percent concentration of 0.1%, and the mobile phase solution B2 being a trifluoroacetic acid acetonitrile solution with trifluoroacetic acid in a volume percent concentration of 0.1%. In the linear gradient elution, the volume percentage of the B2 solution was uniformly increased from 40% to 65%, and the volume percentage of the A2 solution was uniformly decreased from 60% to 35%, with the elution time of 11 minutes, the elution flow rate 1 mL per minute, and the ultraviolet detection wavelength 220 nanometers. The test result of the analytical high performance liquid chromatograph showed that the purity of the mPEG₂₀₀₀₀CY was 94.5%.

### Example 2 Preparation of PEGylated Polypeptide mPEG₄₀₀₀₀CY

The mPEGxMaI (x=40000, i.e., the PEG has an average molecular weight of 40000, mPEG₄₀₀₀₀MaI, a product of Beijing JenKem Technology Co., Ltd.), via a Michael addition reaction, was reacted with the thiol group in the N-end amino acid, i.e. cysteine, of the polypeptide CY, to obtain the mPEG₄₀₀₀₀CY.

The mPEG₄₀₀₀₀CY was prepared according to the method described in the item 1 in step (II) in Example 1, except that the mPEG₂₀₀₀₀MaI was replaced by a mPEG₄₀₀₀₀MaI, and the reaction was performed until the polypeptide was completely reacted.

With an Angilent 1200 reverse high performance liquid chromatograph, the reaction product obtained by the above steps was purified. The model of chromatographic column was Angilent Eclipse XDB-C18 Semi-prep, 5 μm, 9.4×250 mm. The operation of chromatograph was described below: a linear gradient elution, wherein the eluent consisted of a mobile phase solution A2 and a mobile phase solution B2, both of which were described in Example 1. In the linear gradient elution, the volume percentage of the solution B2 was uniformly increased from 30% to 52%, and the volume percentage of the solution A2 was uniformly decreased from 70% to 48%, with the elution time of 11 minutes, the elution flow rate 2.5 mL per minute, and the ultraviolet detection wavelength 220 nanometers. The purified product of PEGylated polypeptide mPEG₄₀₀₀₀CY in a fluffy state was obtained after removing the solvent by freeze-drying.

The characterization result of MALDI-TOF mass spectrum of the mPEG₄₀₀₀₀CY was shown in FIG. 2. The structural formula of the mPEG₄₀₀₀₀CY was represented by formula I, wherein the Cys and the PIBC were linked by a peptide bond formed by the carboxyl group of the Cys and the amino group of the N-end amino acid residue of the PIBC.

The purity analysis of the mPEG₄₀₀₀₀CY was performed as described in Example 1 except that in the linear gradient elution, the volume percentage of the solution B2 was uniformly increased from 20% to 100% and the volume percentage of the solution A2 was uniformly decreased from 80% to 0, with the elution time of 25 minutes. The test result of the analytical high performance liquid chromatograph showed that the purity of the mPEG₄₀₀₀₀CY was 95.3%.

### Example 3 Preparation of PEGylated Polypeptide mPEG₂₀₀₀₀LC

A polypeptide obtained by adding cysteine to the C-end of the PIBC shown by SEQ ID NO. 1 in the sequence table was named as “LC”; the amino acid sequence of the LC was shown by SEQ ID NO. 3 in the sequence table; the polypeptide LC shown by SEQ ID NO. 3 was synthesized by GL Biochem (Shanghai) Ltd.

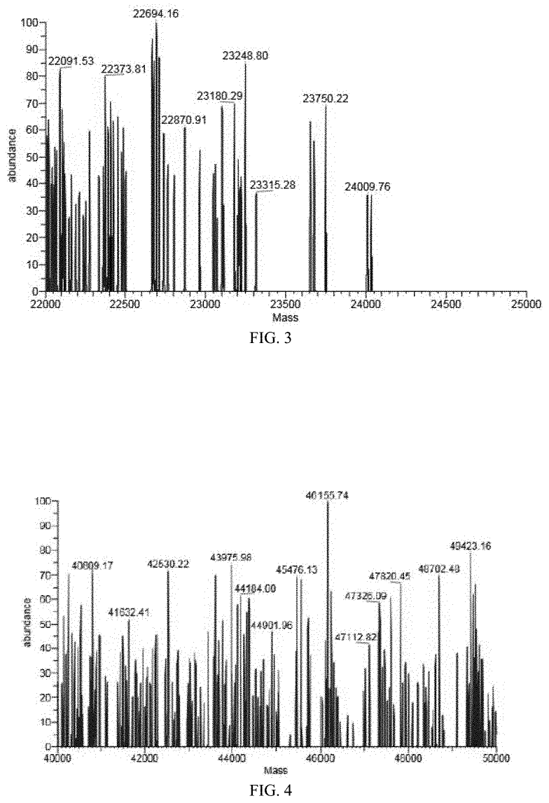

The mPEGxMaI (x=20000, i.e., the PEG has an average molecular weight of 20000, mPEG₂₀₀₀₀MaI, a product of Beijing JenKem Technology Co., Ltd.), via a Michael addition reaction, was reacted with the thiol group in the C-end amino acid, i.e. cysteine, of the polypeptide LC, to produce the mPEG₂₀₀₀₀LC. The procedures for the preparation, purification and characterization of the mPEG₂₀₀₀₀LC were performed as described in Step (II) in Example 1, except that the polypeptide CY in Example 1 was replaced by the polypeptide LC, to obtain a PEGylated polypeptide mPEG₂₀₀₀₀LC in a fluffy state. The MALDI-TOF mass spectrum characterization result of the mPEG₂₀₀₀₀LC was shown in FIG. 3. The test result of the analytical high performance liquid chromatograph showed that the purity of the mPEG₂₀₀₀₀LC was 93.6%. The structural formula of the mPEG₂₀₀₀₀LC was represented by formula I, wherein in the formula I, the Cys and the PIBC were linked by a peptide bond formed by the amino group of the Cys and the carboxyl group of the C-end amino acid residue of the PIBC.

### Example 4 Preparation of PEGylated Polypeptide mPEG₄₀₀₀₀LC

The mPEGxMaI (x=40000, i.e., the PEG has an average molecular weight of 40000, mPEG₄₀₀₀₀MaI, a product of Beijing JenKem Technology Co., Ltd.), via a Michael addition reaction, was reacted with the thiol group in the C-end amino acid, i.e. cysteine, of the polypeptide LC, to obtain the PEGylated mPEG₄₀₀₀₀LC.

The procedures for the preparation, purification and characterization of the mPEG₄₀₀₀₀LC were performed as described in Step (II) in Example 1, except that the mPEGxmaI (x=20000) in Example 1 was replaced by mPEGxmaI (x=40000), and the polypeptide CY in Example 1 was replaced by the polypeptide LC, to obtain a PEGylated polypeptide mPEG₄₀₀₀₀LC in a fluffy state. The MALDI-TOF mass spectrum characterization result of the mPEG₄₀₀₀₀LC was shown in FIG. 4. The test result of the analytical high performance liquid chromatograph showed that the purity of the mPEG₄₀₀₀₀LC was 95.1%. The structural formula of the mPEG₄₀₀₀₀LC was represented by formula I, wherein the Cys and the PIBC were linked by a peptide bond formed by the amino group of the Cys and the carboxyl group of the C-end amino acid residue of the PIBC.

### Example 5 Interactions Between Gp96 Protein and PEGylated Polypeptide

The amino acid sequence of the gp96 protein (human heat shock protein, Genbank Accession NO. AY040226) was shown by SEQ ID NO. 4 of the sequence table, and the coding sequence was shown by SEQ ID NO. 5.

I. Construction of pFastBac™1-Gp96 Plasmid

1. Design and synthesis of primers for gp96: with the sequence of human gp96 genes in GenBank as a template, a forward primer and a reverse primer were designed, wherein the sequence of the forward primer is 5′-CGGGATTCATGGACGATGAAGTTGATGTGGAT-3′ (SEQ ID NO. 6), and the reverse primer sequence is 5′-GCTCTAGATTAGAATTCATCTTTTTCAGCTG-3′ (SEQ ID NO. 7), in which the forward primer contains a BamHI enzyme cutting site at the 5′-end, and the reverse primer contains an Xbal enzyme cutting site at the 5′-end.

2. The mRNA of human liver cancer cell HepG2 was extracted, and cDNA was synthesized by reverse transcription.

3. With the cDNA obtained in step 2 as a template, the target genes were amplified by using the primers designed in step 1 through a Polymerase Chain Reaction (PCR), and a PCR product, i.e., the gp96 gene was obtained.

4. The PCR product obtained in step 3 was cut with two enzymes EcoRI and Xbal, and an enzyme cut product having a size about 2400 bp. was recovered.

5. The pFastBac™1 empty plasmid (Invitrogen, Catalogue No. 10359-016) was cut with two enzymes EcoRI and Xbal, and a backbone carrier having a size about 4700 bp. was recovered.

6. The enzyme cut product having a size about 2400 bp obtained in step 4 and the carrier backbone having a size about 4700 bp obtained in step 5 were linked to obtain a recombinant plasmid, and after being checked by sequencing, the recombinant plasmid with a correct sequence was named as pFastBac™1-gp96. The recombinant plasmid pFastBac™1-gp96 could code and express gp96 protein, and the amino acid sequence of the gp96 protein was shown by SEQ ID NO. 4 in the sequence table.

### II. Purifications of Insect Cell-Expressed Gp96 Recombinant Proteins and Gp96 Protein

The pFastBac™1-gp96 of step I was transfected into Sf9 cells (Invitrogen, Catalogue No. 11496-015) with a Cellfect II reagent (Life technologies, Catalogue No. 10362-100). The Sf9 cells transfected with the plasmid were cultured for 72 h. The observed cytopathic situations indicated that recombinant 1^{st}-generation baculovirus (P1) had been released into a culture medium and a cell supernatant was harvested to obtain the P1 virus. A suitable amount of the P1 was added to Sf9 monolayer (1×10⁶ cells/mL) cells, and cultured at 27° C. for 72 hours. The mixture was centrifuged at 4000 rpm for 5 min, and a supernatant was harvested to obtain 2^{nd}-generation virus (P2). A suitable amount of the P2 was added to 100 mL of Sf9 (1.6×10⁶ cells/mL) suspension cells and was cultured at 27° C., 100-120 rpm/min for 72 hours, to obtain 3^{rd}-generation virus (P3) by amplifications. Rat anti-gp96 antibody (Santa Cruz, product No. sc-56399) was used as a primary antibody to perform Western blotting, and it was indicated that the gp96 protein was expressed in Sf9 cells.

Subsequently, a suitable amount of the P3 virus was added to fresh Sf9 cells (1.5×10⁶ cells/mL, 300 mL) and was subjected to suspension culture at 27° C., 100-120 rpm/min in an Insect-XPRESS™ Protein-free Insect Cells medium with L-Glutamine (Catalogue No. 12-730Q). After 72 hours, the culture medium for the suspension culture was centrifuged at 7000 rpm for 20 minutes to give a clear supernatant which was filtered through a 0.22 mm filtration membrane and then purified by passing through a HiTrap Q HP column and a Superdex 200 10/300 GL ion chromatographic column to give a purified product. The purified product was identified by a denatured polyacrylamide gel electrophoresis and a Western blot immunoblotting test (the used primary antibody is a rat anti-gp96 antibody (Santa Cruz, product No. sc-56399)) and the identification result was shown in FIG. 5, which confirmed that the purified product contained highly pure gp96 protein. With an ultrafiltration tube, the solvent in the above purified product was replaced with a PBS buffer solution, and concentrated, and the protein concentration was measured by using a BCA method. At last, the protein was packaged and stored at −80° C.

### III. Interaction of Gp96 Protein and PEGylated Polypeptide Fragment

The interaction between the fragments of the PEGylated polypeptide prepared in Examples 1-4 and the gp96 protein was respectively evaluated by a Biacore method. The instrument used in this experiment is a BIAcore3000 system with a CM5 sensing chip. According to the specification, the gp96 protein obtained in step II were fixed on the CM5 sensing chip through amino group coupling and the specific method was described below: a filtered and degassed HBS buffer solution (10 mmol/L HEPES, 0.15 mol/L NaCl, 3.4 mol/L EDTA, 0.05% P-20; pH 7.4) was used as a mobile phase solution, and a CM5 sensor chip module was embedded in the BIAcore system; the flow rate through a flow cell was set to be 5 μL/min; a mixed solution of 0.2 mol/L N-ethyl-N-dimethyl-aminopropyl carbodiimide and 0.05 mol/L N-hydroxysuccinimide in equal volumes was used to activate the surface of the CM5 sensing chip for 7 min; 35 μL of 1 mg/mL gp96 protein was injected to the activated surface to make the protein bound with the surface of the CM5 sensor chip; 35 μL of ethanolamine was injected to deactivate excess reactive groups; 10 μL of 20 mmol/L HCl was injected rapidly, and then non-covalently bound materials were removed with Extraclean; by placing a 1^{st} baseline report point before starting the injection of the gp96 protein, and by placing a 2^{nd} baseline report point at 2 min after the completion of the injection of 20 mmol/L HCl, the level of the bound gp96 protein was measured; a flow cell of the bound gp96 protein was set as a test passage, a flow cell of non-bound gp96 protein was set as a reference passage, an HBS buffer solution was used as a mobile phase, and the flow rate of the flow cells was 10 μL/min, the substances to be tested were injected into the gp96 protein flow cell and the reference flow cell simultaneously; the binding reaction was performed at 22-24° C. and in condition of pH 7.4; 10 μl of one PEGylated polypeptide fragment in Examples 1-4 or PIBC (diluted with a HBS buffer solution containing 1 mg/mL carboxymethyl dextran) were injected for test; 10 μL of 20 mmol/L HCl were quickly injected and the surfaces of gp96 protein were regenerated with Extraclean; 10 μL of the PEGylated polypeptide fragments were further injected. This cycle was repeated to determine the reproducibility of the polypeptide fragment bound to the surface of the gp96 protein. According to the above steps, polypeptide fragments in different concentration levels (156, 312, 625, 1250, 2500 nmol/L) were respectively tested, and the determination of each concentration level was repeated once.

The binding coefficients K_{D} (mM/L) of the PEGylated polypeptide fragments in Examples 1-3 to gp96 protein were shown in Table 1.

TABLE 1
Coefficients of binding reaction between
PEGylated polypeptides and gp96 protein

| Polypeptide | Binding coefficient K_{D} (mM/L) |
|---|---|
| mPEG₂₀₀₀₀CY | 4.562 |
| mPEG₄₀₀₀₀CY | 50.41 |
| mPEG₂₀₀₀₀LC | 10.31 |

It was indicated that the PEGylated polypeptides in Examples 1-3 each had an affinity to gp96 protein, and the mPEG₂₀₀₀₀CY obtained by modifying a peptide with PEG having a molecular weight 20000 at the N-end of had the strongest binding ability.

### Example 6 Irritation Test for Sites Injected with PEGylated Polypeptide

A control vehicle group, a PIBC group, a mPEG₂₀₀₀CY group (the PEG having molecular weight of 2000, a polypeptide modified by adding cysteine at N-end, prepared by referring to Example 1), a mPEG₅₀₀₀CY group (the PEG having a molecular weight of 5000, a polypeptide modified by adding cysteine at N-end, prepared by referring to Example 1), a mPEG₂₀₀₀₀CY group, a mPEG₂₀₀₀₀LC group, a mPEG₄₀₀₀₀CY group and a mPEG₄₀₀₀₀LC group were set. In the control vehicle group, a vehicle (physiological saline) was injected to SD rats, and in the other groups PEGylated peptides respectively at a dose of 125, 250, 500 mg/kg (in PIBC), were administrated in a single subcutaneous injection to SD rats, 5 animals in each group. The rats were observed daily for clinical condition and changes at the injected sites, and they were euthanized after 7 days and observed for pathological changes at the injected sites through dissections and by a microscope.

During the test, as compared with the rats in the vehicle group, the rats in the PIBC group, mPEG₂₀₀₀CY group and the mPEG₅₀₀₀CY group had less autonomous activities and reduced body weight and appetite, and the performance was relevant to administration doses; the PIBC, mPEG₂₀₀₀CY and mPEG₅₀₀₀CY had stronger skin irritation to the administration site. The SD rats administrated by single subcutaneous injection at doses of 125, 250 or 500 mg/kg, had skin abnormality (scabbing or bruising) at the administration site, and the irritation became more obvious with the increase of dose. By gross dissection, the skin scabbing at the administration site and adjacent sites thereof can be observed; with a microscope, corresponding histological changes were observed, and at the administration site, local reactions such as denaturing or necrosis occurred.

The clinical manifestations of the mPEG₂₀₀₀₀CY, mPEG₂₀₀₀₀LC, mPEG₄₀₀₀₀CY and mPEG₄₀₀₀₀LC groups were not abnormal, with significantly reduced skin irritations, and at the highest dose of 500 mg/kg, no erythema or edema appeared at the injected sites. Moreover, no obvious abnormality was observed through dissections and by a microscope.

As compared with the PIBC, the mPEG₂₀₀₀₀CY and the mPEG₅₀₀₀₀CY, which showed strong skin irritation and greatly influenced their clinical applications, the polypeptide modified with PEG having a molecular weight of 20000-40000 was remarkably improved in the skin irritation.

### Example 7 Hemolytic Test of PEGylated Polypeptides

20 mL of rabbit blood was taken, and the blood was stirred with a glass rod to remove fibrinogen therein, to produce defibrinated blood. A 0.9% sodium chloride solution in an amount of 10 times of the blood was added, and the solution was shaken up and centrifuged at 1000-1500 r/min for 15 min, and then the supernatant was removed. The precipitated erythrocytes were further washed with 0.9% sodium chloride solutions for 2-3 times according to the above method until the supernatant did not exhibit red color. The resultant red cells were formulated into a 2% suspension solution with 0.9% sodium chloride solution for later use in tests.

Nine clean test tubes were taken and numbered, wherein tubes No. 1-7 were sample tubes (PIBC, mPEG₂₀₀₀CY, mPEG₅₀₀₀CY, mPEG₂₀₀₀₀CY, mPEG₂₀₀₀₀LC, mPEG₄₀₀₀₀CY and mPEG₄₀₀₀₀LC, with a concentration 5 mg/ml based on the PIBC), tube No. 8 was a negative control tube, and tube No. 9 was a positive control tube. According to the amounts shown in the following table, a 2% erythrocyte suspension, a 0.9% sodium chloride solution or distilled water were added in order. After being uniformly mixed, the mixture was immediately placed in an incubator at a temperature 37±0.5° C. for incubation. The samples were observed once every 15 minutes. After 1 hour, they were observed once every 1 hour and the observation went on for 3 hours.

| Serial number of test tube | 1 | 2 | 3 | 4 | 5 | 6 | 7 | 8 | 9 |
|---|---|---|---|---|---|---|---|---|---|
| 2% Erythrocyte | 2.5 | 2.5 | 2.5 | 2.5 | 2.5 | 2.5 | 2.5 | 2.5 | 2.5 |
| suspension (ml) | | | | | | | | | |
| Physiological saline (ml) | 2.0 | 2.0 | 2.0 | 2.0 | 2.0 | 2.0 | 2.0 | 2.5 | 0 |
| Distilled water (ml) | 0 | 0 | 0 | 0 | 0 | 0 | 0 | 0 | 2.5 |
| Test substances (ml) | 0.5 | 0.5 | 0.5 | 0.5 | 0.5 | 0.5 | 0.5 | 0 | 0 |

In the test, with the addition of the test substances PIBC, mPEG₂₀₀₀CY and mPEG₅₀₀₀CY, after 3 hours, the solutions were clear and red, and a small quantity of erythrocytes were left at the bottom of the tubes, indicating that hemolysis occurred. Thus, the PIBC, mPEG₂₀₀₀CY and mPEG₅₀₀₀CY were not suitable for injection use. However, with the addition of the test substances mPEG₂₀₀₀₀CY, mPEG₂₀₀₀₀LC, mPEG₄₀₀₀₀CY and mPEG₄₀₀₀₀LC, after 3 hours, all erythrocytes sunk in the solutions, and the supernatant was colorless and clear, indicating that no hemolysis occurred. It was indicated that the PIBC modified with PEG having a molecular weight of 20000-40000 may be used by injection.

### Example 8 Toxicological Test for Repeated Intravenous Infusion of PIBC or mPEG₂₀₀₀₀CY to SD Rats for 14 Days

Thirty rats (15/sex) were divided into 3 groups in the sex segment according to body weight, and they were administrated with a vehicle control (a sodium chloride injection solution, 1^{st} group), the PIBC at 10 mg/kg/day (2^{nd} group) and the mPEG₂₀₀₀₀CY at 10 mg/kg/day by intravenous infusion, respectively. The administrated dose was 10 mL/kg, and the administration speed was 2 mL/kg/min. Euthanasia was performed at D15 after 14 days of repeated administrations. The animals were observed and recorded for the death, clinical symptoms, body weights and food intake, and they were subjected to clinical pathological tests (blood cell counting test, blood coagulation function index test, and blood biochemical index test), organ weighing, and gross dissection observations. The tissues that were observed to be abnormal were subjected to histopathological examinations.

Vehicle control group: the animals were not observed to be abnormal.

PIBC group: from the third day to the end of the administrations, irritation reactions sequentially occurred at the administration sites, including mild/moderate swelling at tails (M: 5/5, F: 5/5), discoloration (purple) at the distal ends of the tails (M: 5/5, F: 4/5), and mild/moderate/severe ulcerations at injected sites (M: 5/5, F: 2/5). In addition, there were 3/5 of females that showed reduced spontaneous activities at D1 to D3 and 1/5 of females that showed abnormal movements (slow walking) after the administration at D1 and before the administration at D2. Body weights and body weight gains of male animals were reduced at D7. As for blood cell counting, it could be seen that male and female animals had increase in WBC, Neut, Baso, Retic, P L T and Mono, and had decrease in RBC, HGB and HCT In addition, male animals had increase in Lymph (10\hat{ }9/L) and Eos (10\hat{ }9/L) while female animals had increase in Mono. As for the coagulation function index, it could be seen that the male and female animals had prolonged PT and increased FIB. As for the blood biochemical index, it could be seen that the male and female animals had reduced Alb, A/G and Na⁺ and increased CK and LDH. Moreover, the male animals had increased Cre while the female animals had increased AST and ALT. As for gross dissection, it could be seen that 8/10 of animals had enlarged inguinal lymph nodes, 10/10 of animals had swelling at injected sites (tail), 9/10 of animals had discolorations, and 5/10 of animals had skin ulceration at injected sites (tail). Microscopic examinations could reveal changes, such as cortical lymphocyte histiocyte hyperplasia and medullary sinus histiocyte hyperplasia and blooding of inguinal lymph node; at injected sites (tail), crust formation and/or ulcer, epidermal necrosis, hyperplasia and hyperkeratosis, dermal necrosis, edema and inflammatory cell infiltration, subcutaneous tissue necrosis, edema, hyperplasia of fibrous tissues and/or fibroblasts, infiltrations of inflammatory cell with predominant neutrophils, thrombosis, atrophy/necrosis of deep muscle fibers in subcutaneous tissues, reactive hyperplasia of osteoblast and the like. mPEG₂₀₀₀₀CY group: each animal was not observed to die or in dying; the body weights and the food intakes were reduced temporarily; the coagulation function and the blood biochemical index were not seen to be obviously abnormal; and through the gross dissection, each organ was not observed to obviously abnormal.

It was indicated that the intravenous injection administration of the PIBC had strong irritation and toxicity, while the toxicity of mPEG₂₀₀₀₀CY with PEGylation medication was obviously reduced.

### Example 9 Effects of PEGylated Polypeptides on Breast Cancer Cells SKBr3

Breast cancer cell SKBr3 was an ATCC (American type culture collection) product, with the product No. HTB-30. Breast cancer cell MDA-MB-231 was an ATCC (American type culture collection) product, with the product No. HTB-26. The PEGylated polypeptide fragments prepared in Examples 1-4 were subjected to the following experiments:

### I. Inhibition of PEGylated Polypeptide on Proliferation of Breast Cancer Cells SKBr3

With a CCK-8 kit (Dojindo Laboratories, catalogue No. CK 04-05), the inhibitory effect of each PEGylated polypeptide fragment on the proliferation of breast cancer cells SKBr3 was tested. The specific operation steps were described below:

1. SKBr3 cells were spread in 96-well plates with a convergence of about 50%. For each group of cells three wells were provided.
2. After the cells were adhered to the wall, the PEGylated polypeptide (with a final concentration of 6 μM) was added as an experimental group, and three wells without the polypeptide were provided as control groups.
3. At various time points (0, 3, 6, 12 hours), 10 μl of CCK-8 test reagent were added to each well and incubated at 37° C. for 2 hours.
4. OD values were measured at 490 nm.

The cell growth inhibition rate was calculated according to the formula: (OD₄₉₀ average value of control group—OD₄₉₀ average value of experimental group)/OD₄₉₀ of control group×100%. The cell growth inhibition rates (average values) for the respective PEGylated polypeptide fragment treatment group were shown in Table 2.

TABLE 2
Growth inhibition rates of cells treated
with PEGylated polypeptides

| Polypeptide | Inhibition rate |
|---|---|
| mPEG₂₀₀₀₀CY | 33% |
| mPEG₄₀₀₀₀CY | 11% |
| mPEG₂₀₀₀₀LC | 19% |
| mPEG₄₀₀₀₀LC | 3% |

It was indicated that the PEGylated polypeptides prepared in Examples 1-4 each could inhibit proliferation (growth) of the breast cancer cells SKBr3, and the mPEG₂₀₀₀₀CY had the most remarkable inhibitory effect.

### II. Inhibition of PEG Polypeptides on Invasiveness of Breast Cancer Cells MDA-MB-231

With Tanswell plate (Corning, product No. 3422) and Matrigel (BD, product No. 354234), influences of the PEGylated polypeptides prepared in Examples 1-4 on the invasiveness of breast cancer cells MDA-MB-231 were evaluated respectively. The cell invasion experiments were performed according to Transwell and Matrigel specifications. The main operation steps were described below:

1. At the day before the experiments, the Matrigel was frozen and thawed overnight on ice, and 60 μl of the Matrigel were added to the upper chamber of the Transwell, coated at 37° C. for 1 hour, and washed 2 times with a PBS buffer solution.
2. Digested and counted breast cancer cells MDA-MB-231 were diluted to 0.4 million/ml with a serum-free culture solution (a final concentration of 10 μM) containing the PIBC or the PEGylated polypeptide prepared in Examples 1, 2, 3 or 4. For an experimental group, 100 μl of the diluted solution were added to the upper chamber of the Transwell, and 600 μl of a complete cell culture solution were added to the lower chamber of the Transwell. For a negative control group, a serum-free culture solution was used to replace the serum-free culture solution containing the polypeptide fragment.
3. The incubation was continued at 37° C. for 24 hours in a CO₂ incubator with 5% CO₂. The cells in the upper chamber of the Transwell were scraped off with a cotton swab, and the culture solution was fixed with 50% methanol/50% acetone for 15 minutes, and washed 3 times with PBS buffer solutions. DAPI was used for mounting, and the number of invaded cells were counted with a fluorescent microscope.

The invasion inhibition rate was calculated according to the formula: the number of invading cells in negative control group—the number of invading cells in experimental group)/the number of invading cells in negative control group×100%.

The inhibition rates (average values) of groups treated by PEGylated polypeptide fragments relative to the negative control group against MDA-MB-231 invasion were shown in Table 3.

TABLE 3
Inhibition rates of PEGylated polypeptide fragment
treatment groups relative to the negative control
group against MDA-MB-231 invasion

| Polypeptide | Inhibition rate |
|---|---|
| mPEG₂₀₀₀₀CY | 53.8% |
| mPEG₄₀₀₀₀CY | 21.2% |
| mPEG₂₀₀₀₀LC | 26.3% |
| mPEG₄₀₀₀₀LC | 5.2% |

It was indicated that the PEGylated polypeptides prepared in Examples 1-4 could inhibit the invasions of breast cancer cells MDA-MB-231, and the mPEG₂₀₀₀₀CY had the most obvious inhibitory effect.

### III. Promotion of PEGylated Polypeptide on Apoptosis of Breast Cancer Cells SKBr3

The effect of the PEGylated polypeptides prepared in Examples 1-4 for promoting the apoptosis of breast cancer cells SKBr3 were respectively evaluated. The specific steps were described as follows:

1. Breast cancer cells SKBr3 were inoculated in a 6-well cell culture plate at 0.2 million cells/well.
2. For an experimental group, after the cells were adhered to the wall, the PIBC or the PEGylated polypeptide (a final concentration 6 μM) prepared in Examples 1, 2, 3 or 4 was added and further cultured for 24 hours. For a negative control group, a PBS solution was added to replace the PIBC or the PEGylated polypeptide.
3. With a Vybrant® Apoptosis Assay kit produced by Invitrogen, the cells were dyed and analyzed by a flow cytometer. The specific operation steps were described below:
(1) Cells were routinely digested with a pancreatic enzyme and washed twice with PBS buffer solutions (it was suitable that the cell number typically is equivalent to one quarter of a 6-well plate or a 24-well plate).
(2) The cells were gently suspended with 20 μl of 1× Annexin V Buffer and after 1 μl of FITC Annexin V was added, they were gently mixed to be uniform. The cells were dyed at room temperature in a dark place for 15 minutes.
(3) To the reaction tube, 1× Annexin V Buffer was added to make the final volume 200 μl.
(4) PI with the concentration 100 μg/ml was added to ensure that the final concentration was 1 μg/ml, and the cells were dyed for about 3 minutes at room temperature in a dark place and then tested.

The increase of apoptosis rates (average values) of the groups treated with the PEGylated polypeptides in Examples 1-4 relative to the negative control group were shown in Table 4.

TABLE 4
Increase of apoptosis rates of PEGylated polypeptide
treatment groups relative to negative control group

| Polypeptide | Apoptosis Rate |
|---|---|
| mPEG₂₀₀₀₀CY | 32.1% |
| mPEG₄₀₀₀₀CY | 11.1% |
| mPEG₂₀₀₀₀LC | 21.1% |
| mPEG₄₀₀₀₀LC | 3.8% |

It was indicated that the PEGylated polypeptides prepared in Examples 1-4 can promote apoptosis of the breast cancer cells SKBr3, and the mPEG₂₀₀₀₀CY had the most obvious promotion effect.

IV. Inhibition of the mPEG₂₀₀₀₀CY on Growth of Transplanted Tumor of Breast Cancer Cells MDA-MB-231

Inhibitory effects of the mPEG₂₀₀₀₀CY on the growth of transplanted tumor of the breast cancer cells MDA-MB-231 were evaluated. The specific steps were described below:

1. Breast cancer cells MDA-MB-231 that were cultured to the logarithmic growth phase were subcutaneously inoculated into BALB/c nude mice (Beijing Vital River Laboratory Animal Technology Co., Ltd.), each being inoculated with 100 million cells, to establish a transplantation tumor model, and then the tumor cells in the nude mice were subjected to passages 3 times, for use in tumor inoculation experiments.
2. When the tumor grew to 100 mm³, the BALB/c nude mice were randomly divided into 3 groups, each group with 5 mice, and they were subjected to the following therapy treatments, the day of the first treatments being recorded as 1^{st} day.

PIBC group: an intravenous injection therapy was performed with a PIBC solution (polypeptide dissolved in 0.9% physiological saline) at a dose of 5 mg/kg per injection, administered once a day, and treated totally for two weeks, mPEG₂₀₀₀₀CY group: an intravenous injection therapy was performed with the mPEG₂₀₀₀₀CY solution (mPEG₂₀₀₀₀CY dissolved in 0.9% physiological saline) at a dose of 5 mg/kg (calculated based on the amount of the PIBC), with the same injection volume as that of the PIBC treatment group, administered once every two days, and treated totally for two weeks;

control group: an intravenous injection therapy was performed with a PBS buffer solution, with the same injection volume as that of the PIBC treatment group, administrated once a day, and treated totally for two weeks.
3. The volume of the tumor was examined twice every week. After a 2-week therapy, the nude mice were sacrificed, and the tumors were weighed and the tumor inhibition rates were calculated.

The tumor inhibition rate was calculated according to the formula: (tumor volume of mice in control group—tumor volume of mice in polypeptide treatment group)/tumor volume of mice in control group×100%.

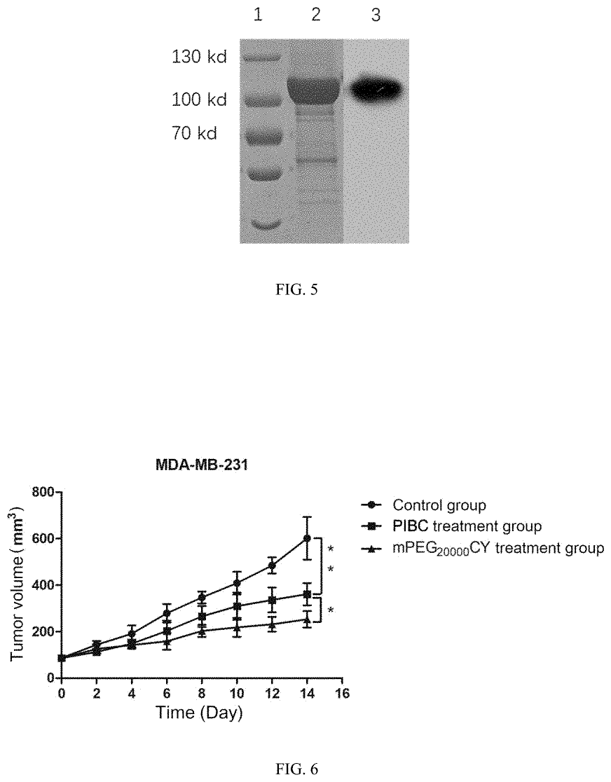

The results for the tumor inhibition rates of the PIBC and mPEG₂₀₀₀₀CY treatment groups were shown in FIG. 6. It was indicated that both the PIBC and the mPEG₂₀₀₀₀CY can effectively inhibit the growth of breast cancer tumor caused by breast cancer cells MDA-MB-231.

### Example 10 Evaluation on Plasma Half-Life of PEGylated Polypeptides in SD Rats

Test polypeptides: PIBC, mPEG₂₀₀₀₀CY

### 1. Plotting Standard Curves

A borax buffer solution (pH 9.5) was used to formulate stock solutions of the PIBC and the mPEG₂₀₀₀₀CY at a concentration 1 mg/mL. A proper amount of the stock solution was taken and formulated with an acetonitrile aqueous solution having the volume percentage 50% of acetonitrile into standard curve working solutions with polypeptide concentrations of 25, 37.5, 50, 75, 100, 150 and 250 μg/mL. 20 μl of the prepared standard curve working solutions were taken and added with 80 μl of blank mouse plasma, to formulate standard curve samples with polypeptide concentrations of 5, 7.5, 10, 15, 20, 30 and 50 μg/mL. 20 μl of a 20% (mass percent) phosphoric acid solution and 300 μl of methanol-acetonitrile (the volume ratio of methanol to acetonitrile is 1:1) were added into the standard curve samples and uniformly mixed for about 2 min by vortex; the solution was centrifuged at 4000 rmp/min for 10 min, and the supernatant was taken as a sample for analyses, to obtain the standard curves of the respective test polypeptides. According to the method, the quality-controlled samples were formulated and the precision degree was measured.

### 2. Experimental Procedures

Drug formulating: before the administration, the formulation was performed. The test polypeptide was dissolved into a uniform and transparent solution with a 0.9% sodium chloride injection solution and a 5 mM Na₂HPO₄ solution in equal volume, wherein the final concentrations of the PIBC and the mPEG₂₀₀₀₀CY were 8 mg/ml and 12 mg/ml respectively, and the solutions were used for intravenous administration.

Test animals: male and female SD rats, with body weight of 160-180 g and provided by Beijing Huafukang Biotechnology Co., Ltd.

Animal experiments: administration: each polypeptide was used to treat four SD rats, with two in male and two in female. Before the administration, the rats were weighed, and the administration dose was 8 mg/kg.

Sample collecting: the time at the administration was recorded as zero time, and in the PIBC administrated group, blood was taken from tail vein at zero time and at 2 min, 10 min, 20 min, 40 min, 60 min, 90 min and 120 min after administrations. In the mPEG₂₀₀₀₀CY administrated group, blood was taken from tail vein at time zero and at 30 min, 1 h, 2 h, 4 h, 6 h, 10 h, 12 h, 24 h, 36 h, 48 h and 72 h after administrations. 0.3 mL of blood were taken each time and charged into a centrifuge tube filled with 6 μl of aprotinin and 5 μl of heparin sodium, and then centrifuged at 4500 rmp/min for 5 min. The supernatant blood plasma was separated and stored in a refrigerator at −80° C.

Sample treatment: 100 μl of test sample plasma were taken and added with 20 μl of a 20% phosphoric acid solution, 20 μl of a 50% acetonitrile aqueous solution and 300 μl of a methanol-acetonitrile (1:1) solution, and they were uniformly mixed by vortex for about 2 min and centrifuged at 4000 rmp/min for 10 min. The supernatant was taken as the sample for analysis.

Chromatographic conditions: chromatographic column: XSSELECT CSH C18, 4.6×150 mm, 5 μm; mobile phase: phase A: 0.1% (volume percent) TFA aqueous solution, phase B: 0.1% (volume percentage) TFA acetonitrile solution; an eluent consisting of phase A and phase B, wherein the volume percentage of phase B in the eluent was uniformly increased from 20% to 35%, and the volume percentage of phase A was uniformly decreased from 80% to 65%; elution time: 10 minutes; elution flow rate: 1 mL per minute; ultraviolet detection wavelength: 220 nm; injection volume: 20 μL.

### 3. Experimental Results

1) The relation equations between the drug concentration and the peak area obtained from the standard curves of the PIBC and mPEG₂₀₀₀₀CY were described below: y=2.879x+4.12 (R=0.996) and y=3.742x+0.98 (R=0.991), respectively, wherein y is the peak area and x is the drug concentration.
2) The concentration of each drug at each time point was obtained according to the standard curves, and the results were shown in Table 5.

TABLE 5
Blood drug concentrations at different times

| | | Blood drug | | | Blood drug |
|---|---|---|---|---|---|
| Test | Time | concentration | | Time | concentration |
| sample | (min) | (ng/ml) | Test sample | (h) | (ng/ml) |
| PIBC | 2 | 2324.5 | mPEG₂₀₀₀₀CY | 0.5 | 720.2 |
| PIBC | 10 | 1137.2 | mPEG₂₀₀₀₀CY | 1 | 704.8 |
| PIBC | 20 | 896.3 | mPEG₂₀₀₀₀CY | 2 | 650.1 |
| PIBC | 40 | 264.7 | mPEG₂₀₀₀₀CY | 4 | 602.2 |
| PIBC | 60 | 247.8 | mPEG₂₀₀₀₀CY | 6 | 541.8 |
| PIBC | 90 | 116.1 | mPEG₂₀₀₀₀CY | 10 | 519.3 |
| PIBC | 120 | 38.1 | mPEG₂₀₀₀₀CY | 12 | 496.5 |
| PIBC | | | mPEG₂₀₀₀₀CY | 24 | 434.5 |
| | | | mPEG₂₀₀₀₀CY | 36 | 233.9 |
| | | | mPEG₂₀₀₀₀CY | 48 | 125.4 |
| | | | mPEG₂₀₀₀₀CY | 72 | 59.3 |

It was indicated from the results in Table 5 that: the half-life (T_{1/2}) of the mPEG₂₀₀₀₀CY in animal bodies was about 30.1 h, which was prolonged by 80 times as compared with the half-life 22 min of the PIBC.

The above disclosures are only preferred examples of the invention, but not for limiting the invention. Any modifications, equivalents, improvements and the like made within the spirits and rules of the invention should be encompassed in the protection scope of the invention.

1. A PEGylated polypeptide, comprising a PIBC and a PEG having an average molecular weight of about 20000-40000, the PIBC being covalently linked to the PEG, wherein the PIBC is selected from:
A1) a polypeptide having an amino acid sequence shown by SEQ ID NO. 1; and
A2) a polypeptide derived from A1) and having the same functions as A1), with an amino acid sequence, as compared with the amino acid sequence shown by SEQ ID NO. 1, with replacements and/or deletions and/or additions of one or more amino acid residues or with at least 60%, 80%, 85%, 90%, 95%, 96%, 97%, 98% or 99% identity.

2. The PEGylated polypeptide of claim 1, wherein the PIBC and the PEG are covalently linked via a linking group, or directly linked via a covalent bond.

3. The PEGylated polypeptide of claim 1, wherein the covalent bond linking is accomplished by performing a Michael addition reaction with Reactant 1 and Reactant 2, wherein Reactant 1 is the PEG linked to maleimide at one end, and Reactant 2 is the PIBC with a thiol-containing amino acid residue at the N- or C-end.

4. The PEGylated polypeptide of claim 3, wherein the Reactant 1 has a structure represented by formula II: 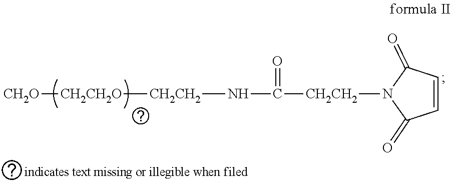
in the formula II, n is the polymerization degree of the PEG.

5. The PEGylated polypeptide of claim 3, wherein the thiol-containing amino acid residue is a cysteine residue.

6. A method of preparing the PEGylated polypeptide of claim 1, comprising the step of covalently linking a PIBC and a PEG having an average molecular weight of 20000-40000.

7. A pharmaceutical composition comprising the PEGylated polypeptide of claim 1.

8. A formulation comprising the PEGylated polypeptide of claim 1.

9. A method of treating and/or preventing a disease associated with overexpression of gp96 protein in a subject in need thereof, comprising administering a therapeutically and/or prophylactically effective amount of the PEGylated polypeptide of claim 1 to the subject.

10. A method of inhibiting proliferation and/or growth and/or invasion of a tumor cell, promoting apoptosis of a tumor cell, and/or inhibiting tumor growth, comprising administering the PEGylated polypeptide of claim 1 to the tumor cell or tumor.

11. The PEGylated polypeptide of claim 1, wherein the PEG is linked to the N-end or C-end of the PIBC, and/or
wherein the PEG is a linear PEG or a branched PEG.

12. The PEGylated polypeptide of claim 2, wherein the PEGylated polypeptide has the following structure:
R—(CH₂CH₂O)ₙ-linker-PIBC;
wherein n is the polymerization degree of the PEG, and the n meets the condition that the PEG has a molecular weight of about 20000-40000; R is the end group of the PEG, such as methoxy; “linker” is a linking group, e.g., 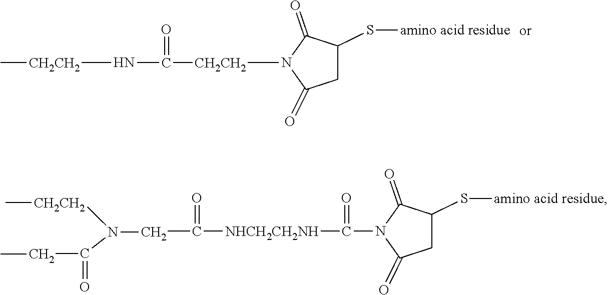
wherein the amino acid residue is an amino acid (e.g., cysteine (Cys)) residue with a thiol group.

13. The PEGylated polypeptide of claim 2, wherein the PEGylated polypeptide has a structure represented by formula I: 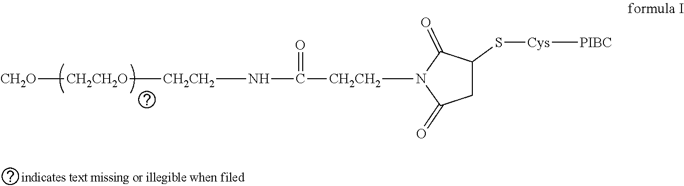
in the formula I, n is a polymerization degree of the PEG.

14. The PEGylated polypeptide of claim 3, wherein the Reactant 2 is a polypeptide with an amino acid sequence shown by SEQ ID NO. 2 or SEQ ID NO. 3.

15. The method of claim 6, wherein the method comprises a step of performing a Michael addition reaction with Reactant 1 and Reactant 2; wherein
the Reactant 1 is a PEG linked to maleimide at one end; and
the Reactant 2 is a PIBC with a thiol-containing amino acid residue at the N- or C-end.

16. The method of claim 9, wherein disease is a tumor.

17. The method of claim 16, wherein the tumor is breast cancer.

18. The method of claim 16, wherein the tumor is triple negative breast cancer.

19. The method of claim 10, wherein the tumor is breast cancer.

20. The method of claim 19, wherein the tumor is triple negative breast cancer.


SEQUENCE LISTING (7 sequences)

SEQ ID NO: 1 - protein, 37 aa, Artificial Sequence
  Synthetic polypeptide  amino acid sequence of PIBC
LNVSRETLQQHKLLKVIRKKLVRKTLDMIKKIADDKY

SEQ ID NO: 2 - protein, 38 aa, Artificial Sequence
  Synthetic polypeptide amino acid sequence of the peptide CY
CLNVSRETLQQHKLLKVIRKKLVRKTLDMIKKIADDKY

SEQ ID NO: 3 - protein, 38 aa, Artificial Sequence
  Synthetic polypeptide amino acid sequence of the peptide LC
LNVSRETLQQHKLLKVIRKKLVRKTLDMIKKIADDKYC

SEQ ID NO: 4 - protein, 782 aa, Homo sapiens
  Amino acid sequence of the gp96 protein
DDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSSSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFIPDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEF

SEQ ID NO: 5 - DNA, 2349 bp, Homo sapiens
  Coding sequence of the gp96 protein
gacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcgtcttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcccagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaggagacagcagaagacacaacagaagacacagagcaagacgaagatgaagaaatggatgtgggaacagatgaagaagaagaaacagcaaaggaatctacagctgaaaaagatgaattctaa

SEQ ID NO: 6 - DNA, 32 bp, Artificial Sequence
  Primer
cgggattcatggacgatgaagttgatgtggat

SEQ ID NO: 7 - DNA, 31 bp, Artificial Sequence
  Primer
gctctagattagaattcatctttttcagctg